(19) United States
(12) Patent Application Publication
(10) Pub. No.: US 2007/0179459 A1
(43) Pub. Date: Aug. 2, 2007

(54) Vertebral body aspirator

(76) Inventors: Fred Geisler, Aurora, IL (US); Eric Major, Purcellville, VA (US); Richards W. Woods, Catonsville, MD (US); Kevin R. Strauss, Columbia, MD (US); Larry McClintock, Gore, VA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/654,798

(22) Filed: Jan. 18, 2007

Related U.S. Application Data
(60) Provisional application No. 60/759,488, filed on Jan. 18, 2006.

(51) Int. Cl. A61M 1/00

(52) U.S. Cl. 604/317

(57) ABSTRACT

A vertebral body aspirator comprises an inner member including a cannula disposed therethrough, the inner member having an opening at a distal end thereof and a rotatable knob at a proximal end thereof, an outer member having a cannula disposed therethrough, the cannula of the outer member being configured and dimensioned for receiving the inner member. The outer member has an aperture located at a distal end thereof. The knob is configured to rotate inner member with respect to the outer member and expose the opening of the inner member. The outer member may include an extended tip located at the distal end thereof. A cartridge may be disposed within the inner member. The cartridge can be disposable and can have a straight-tip or bent-tip at its distal end. The vertebral body aspirator may also include a syringe connected to the proximal end of the cartridge.

### CROSS-REFERENCE TO RELATED APPLICATION

This application claims the benefit of U.S. Provisional Application to Fred Geisler, et al., entitled “Vertebral Body Aspirator,” U.S. Application Ser. No. 60/759,488, filed Jan. 18, 2006.

### BACKGROUND

1. Technical Field

The present disclosure relates generally to orthopedic spine surgery and in particular to devices and methods for vertebral body aspiration.

2. Background of the Related Art

The present disclosure relates generally to orthopedic spine surgery and in particular to a unique vertebral body aspirator device to collect blood and bone marrow from inside a vertebral body.

The spinal column is a complex system of bones and connective tissues that provides support for the human body and protection for the spinal cord and nerves. The adult spine is comprised of 24 vertebral bodies that are subdivided into three areas including seven cervical vertebrae, twelve thoracic vertebrae and five lumbar vertebrae. An intervertebral disc located between each vertebral body cushions and dampens the various translational and rotational forces exerted upon the spinal column.

A spinal column may experience various disorders, diseases and types of injuries during a lifetime. The problems may include, but are not limited to, scoliosis, kyphosis, excessive lordosis, spondylolisthesis, slipped or ruptured discs, degenerative disc disease, vertebral body fracture and tumors. Persons suffering from any of the above conditions typically experience extreme or debilitating pain and, often, diminished nerve function.

A common solution to the above-mentioned conditions involves a surgical procedure known as spinal fusion. A spinal fusion entails fusing two or more vertebral bodies to eliminate motion at the intervertebral disc or joint. During spinal fusion, natural or artificial bone, along with a spacing device, replaces part or the entire intervertebral disc to form a rigid column of bone, and mechanical hardware is connected to the adjacent vertebrae to stabilize the spine in that area, while the bone grows and the fusion takes place.

The mechanical hardware used to immobilize the spinal column typically involves a series of bone screws and metal rods or plates. When spine surgery is performed using a posterior approach, it is common practice to place pedicle bone screws into the vertebral bodies and then connect a metal rod between the bone screws thus creating a rigid structure between adjacent vertebral bodies. When the spine surgery is performed using an anterior approach, it is common practice to attach a metal plate directly to the vertebral bodies and secure it to each vertebral level using one or more bone screws. Immobilization of the spine allows the bone graft time to grow and form a solid column of bone between the respective vertebral bodies.

A major challenge of spine surgery is achieving the bone growth and subsequent fusion from one vertebral body to the next. Standard spine surgery protocol calls for inserting a rigid body between two vertebral bodies to maintain the disc height. The rigid body or interbody is typically pre-packed with autograft or allograft bone. Autograft bone is generally harvested from the patient's iliac crest and is typically the site of long term post-operative pain and discomfort. Allograft bone can be purchased from a third party vendor but since it is not the patient's own bone, it does not perform as well. Other methods for improving bone growth between the vertebral bodies include blood products, graft extenders and bone-morphogenetic proteins (BMP). These other methods are designed to stimulate bone growth by introducing bone growth factors and can be used as graft replacements.

More recently, progenitor cells have been found to be an effective growth factor for bones. These cells can be harvested from the blood and bone marrow in the patient's vertebral bodies, where they are in abundance, during spine fusion surgery just prior to the bone screw insertion. Aside from serving as a substitute autograft source, progenitor cells effectively eliminate the need for iliac crest bone harvesting. The progenitor cells have been found to be as effective in supporting bone growth as the “gold standard” iliac crest autograft but without the residual pain and discomfort.

Since the use of vertebral body progenitor cells is relatively new, collection instruments are not widely available. Several kinds of bone biopsy needles are available for autograft collection at the iliac crest site but these instruments do not perform as well at the vertebral body site. If a bone biopsy needle or a similar device is used in the vertebral body, the open cannula design draws blood and bone marrow up the tube where it spills out the top. Further, a bone biopsy needle works by wedging a sample of bone in its tube and is not designed to collect a fluid so it has no reservoir.

For at least these reasons, there is a need for a surgical instrument capable of breaking through the hard cortical wall of the vertebral body to gain access to the cancellous bone and marrow in its interior. The instrument must also avoid drawing blood through its cannula prematurely and spilling it out through the proximal end. There is also a need for an instrument capable of stopping and restarting blood and marrow collection to allow a surgeon to change the site of collection or the collection reservoir. By combining all these features in one device, a sufficient amount of progenitor cells can be efficiently collected from a patient's vertebral bodies, and used to promote a successful spine fusion.

### SUMMARY

The present disclosure is directed to devices, systems and methods for vertebral body aspiration to perform orthopedic spine surgery. These allow the surgeon to find the vertebral body pedicle using a pedicle probe or any other suitable instrument known in the art. By using the pedicle probe or any suitable equivalent, the surgeon can enter the vertebral body where the bone marrow lies. The device has a cannula and may have a disposable cartridge disposed therein. The proximal end of the cartridge may be equipped with a luer lock to which a standard syringe can be attached. Alternatively, the cartridge itself may have a sufficient reservoir to hold the collected bone marrow. Then, by rotating the device's split handle, the inner member of the device will rotate with respect to the outer member thereby opening the distal end. The vacuum created within the cartridge will draw the bone marrow into the opening, up the cannula and into the collection reservoir.

After the desired amount of bone marrow is collected, i.e., approximately 1 cc per aspiration site, the device may be counter-rotated to close the opening at its distal end. Additionally, various styles of cartridge distal tips can be passed down the cannula, including, but not limited to, a bent tip. When using a bent tip cannulated cartridge, the distal tip may be passed through the distal tip opening of the device to access different areas of bone marrow within the vertebral body. Further, the device may be inserted deeper into the vertebral body to collect more bone marrow.

Another embodiment of the present disclosure includes a device that can be translationally actuated. In operation, once the device penetrates the pedicle, a surgeon may translate the inner member. The inner member is translated farther into the vertebral body while the device's outer member is maintained in the same position. By translating the inner member along its longitudnal axis, the distal tip opening will be revealed. Once again, a straight or bent cannulated cartridge may be passed down said device for bone marrow collection.

At any time, prior to removing said cannulated cartridge, the device's distal tip opening can be closed via rotation or translation such that blood and bone marrow do not flow up the device and spill out in the proximal end thereof. The vertebral body aspirator device may be packaged as a separate system including a series of disposable cannulated cartridges with various styles of distal tips. Finally, upon successful aspiration of bone marrow from said vertebral body, a pedicle bone screw may be inserted into the hole for subsequent spinal fixation.

### BRIEF DESCRIPTION OF THE DRAWINGS

Embodiments of the present invention are disclosed herein with reference to the accompanying drawings, wherein:

FIG. 1A is a side view of a rotatably actuated vertebral body aspirator according to an embodiment of the present disclosure;

FIG. 1B is a front view of the rotatably actuated vertebral body aspirator of FIG. 1A;

FIG. 1C is a perspective view of the rotatably actuated vertebral body aspirator of FIGS. 1A and 2A.

FIG. 2A is a side view of an inner member of a rotatably actuated vertebral body aspirator according to an embodiment of the present disclosure;

FIG. 2B is a front view of the inner member of the rotatably actuated vertebral body aspirator of FIG. 2A;

FIG. 2C is a top view of the inner member of the a rotatably actuated vertebral body aspirator of FIGS. 2A and 2B;

FIG. 2D is a perspective view of the inner member of the rotatably actuated vertebral body aspirator of FIGS. 2A-2C;

FIG. 3A is a side view of the outer member of a rotatably actuated vertebral body aspirator according to an embodiment of the present disclosure;

FIG. 3B is a front view of the outer member of the rotatably actuated vertebral body aspirator of FIG. 3A;

FIG. 3C is a top view of the outer member of the rotatably actuated vertebral body aspirator of FIGS. 3A and 3B;

FIG. 3D is a perspective view of the outer member of the rotatably actuated vertebral body aspirator of FIGS. 3A-3C;

FIG. 4A is a side view of a closed translationally actuated vertebral body aspirator according to an embodiment of the present disclosure;

FIG. 4B is a front view of a closed translationally actuated vertebral body aspirator of FIG. 4A;

FIG. 4C is a perspective view of the closed translationally actuated vertebral body aspirator of FIGS. 4A and 4B;

FIG. 5A is a side view of an open translationally actuated vertebral body aspirator according to an embodiment of the present disclosure;

FIG. 5B is a front view of the open translationally actuated vertebral body aspirator of FIG. 5A;

FIG. 5C is a perspective view of the open translationally actuated vertebral body aspirator of FIGS. 5A and 5B;

FIG. 6A is a front view of an outer member of a translationally actuated vertebral body aspirator according to an embodiment of the present disclosure;

FIG. 6B is a cross-sectional view of the outer member of the translationally actuated vertebral body aspirator, as taken through axis A-A;

FIG. 6C is a top view of the outer member of the translationally actuated vertebral body aspirator of FIGS. 6A and 6B;

FIG. 6D is a perspective view of the outer member of the translationally actuated vertebral body aspirator of FIGS. 6A-6C;

FIG. 7A is a side view of an inner member of a translationally actuated vertebral body aspirator according to an embodiment of the present disclosure;

FIG. 7B is a front view of the inner member of the translationally actuated vertebral body aspirator of FIG. 7A;

FIG. 7C is a top view of the inner member of the translationally actuated vertebral body aspirator of FIGS. 7A and 7B;

FIG. 7D is a perspective view of the inner member of the translationally actuated vertebral body aspirator of FIGS. 7A-7C;

FIG. 8A is a front view of a straight-tip cartridge for use with a vertebral body aspirator according to an embodiment of the present disclosure;

FIG. 8B is a side view of the straight-tip cartridge of FIG. 8A;

FIG. 8C is a perspective view of the straight-tip cartridge of FIGS. 8A and 8B;

FIG. 9A is a front view of a bent-tip cartridge for use with a vertebral body aspirator according to an embodiment of the present disclosure;

FIG. 9B is a side view of the bent-tip cartridge of FIG. 9A;

FIG. 9C is a perspective view of the bent-tip cartridge of FIGS. 9A and 9B;

FIG. 10A is a side view of a rotatably actuated vertebral body aspirator with an extended distal tip according to an embodiment of the present disclosure;

FIG. 10B is a front view of the rotatably actuated vertebral body aspirator with an extended distal tip of FIG. 10A;

FIG. 10C is a perspective view of the rotatably actuated vertebral body aspirator with an extended distal tip of FIGS. 10A and 10B;

FIG. 11A is a front view of a cartridge with a proximal reservoir according to an embodiment of the present disclosure;

FIG. 11B is a perspective view of the cartridge of FIG. 11A;

FIG. 12A is a side view of an assembly including a rotatably actuated vertebral body aspirator and a cartridge with a proximal reservoir according to an embodiment of the present disclosure;

FIG. 12B is a front view of an assembly including the rotatably actuated vertebral body aspirator and the cartridge with a proximal reservoir of FIG. 12A;

FIG. 12C is a perspective view of an assembly including the rotatably actuated vertebral body aspirator and the cartridge with a proximal reservoir of FIGS. 12A and 12B;

FIG. 13A is a top view of a rotatably actuated vertebral body aspirator with an automatic syringe actuator according to an embodiment of the present disclosure;

FIG. 13B is a front view of the rotatably actuated vertebral body aspirator with an automatic syringe actuator of FIG. 13A; and

FIG. 13C is a side view of a portion of the rotatably actuated vertebral body aspirator with an automatic syringe actuator of FIGS. 13A and 13B.

### DETAILED DESCRIPTION OF THE EMBODIMENTS

Embodiments of the presently disclosed vertebral body aspirator are now described in detail or corresponding elements in each of the several views. As used herein, the term “distal” refers to that portion of the vertebral body aspirator, or component thereof, farther from the user while the term “proximal” refers to that portion of the vertebral body aspirator, or component thereof, closer to the user. Terms such as “above,” “below,” “forward,” “rearward,” etc. refer to the orientation of the figures or the direction of components and are simply used for convenience of description.

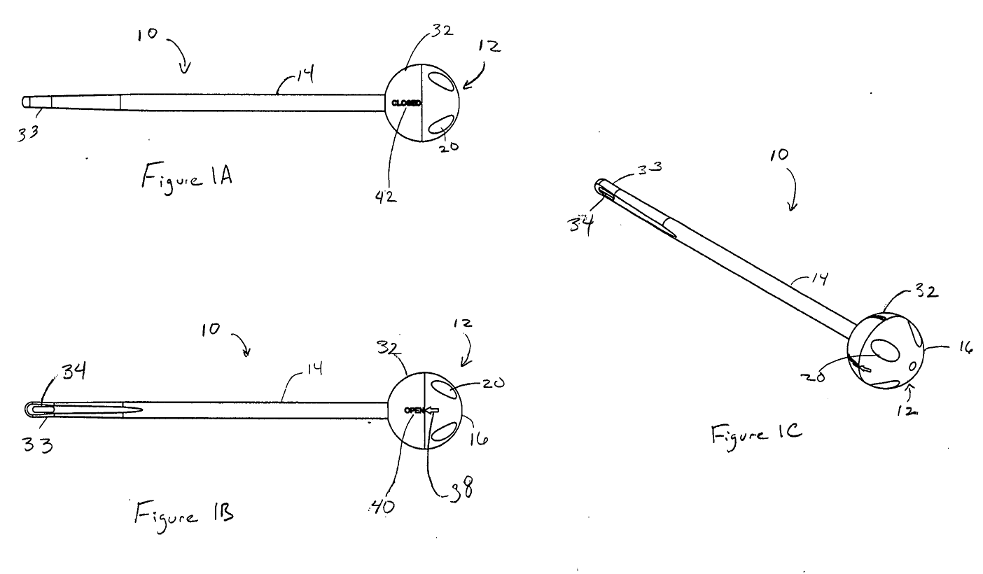

An embodiment of a rotatably actuated aspirator in accordance with the present disclosure is shown in FIGS. 1A-1C as 10. Aspirator 10 includes an inner member 12 having a knob 16, and an outer member 14 having a tip 33 positioned at the proximal end thereof. Tip 33 of outer member 14 includes an opening 34. Inner member 12 is partially disposed within outer member 14 and can be rotated with respect to outer member 14.

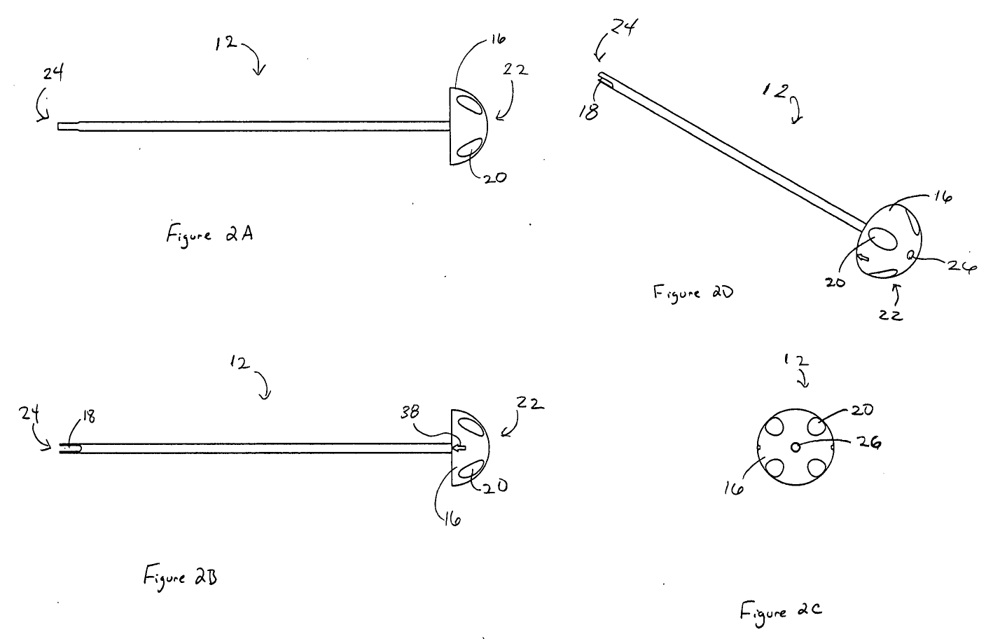

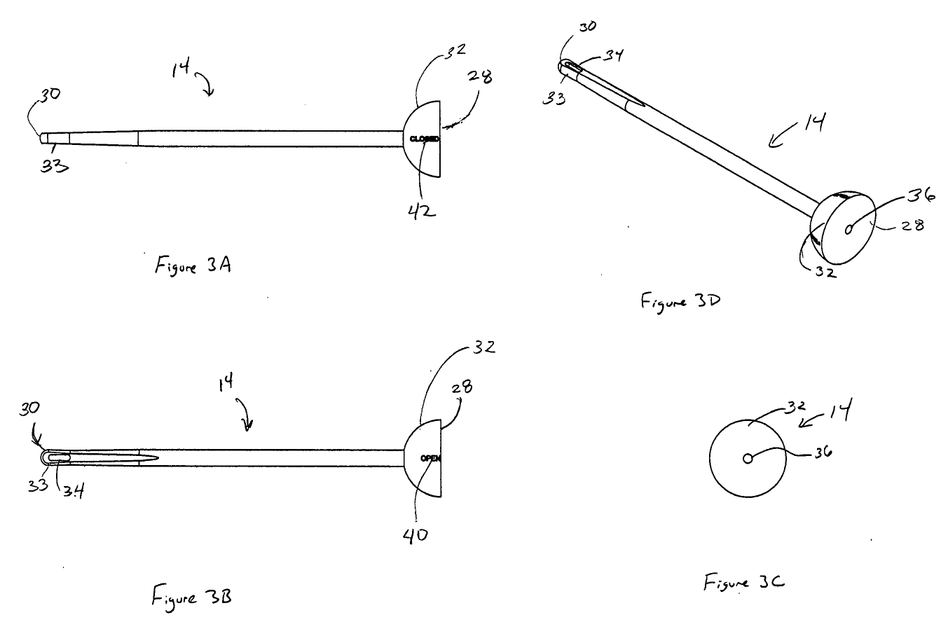

Referring now to FIGS. 2A-2C, inner member 12 of the aspirator 10 has a proximal end 22 and a distal end 24. A knob 16 is attached to the proximal end 22 of inner member 12 and may have at least one indent 20 ergonomically designed for comfortable manipulation. An aperture 18 is situated at the distal end 24 of inner member 12.

In addition, inner member 12 includes a cannula 26 disposed therethrough. Cannula 26 is dimensioned for receiving a cannulated cartridge. Many kinds of cartridges may be used with aspirator 10. For instance, the cartridge may be in the form of a pipette. Further, aspirator 10 may include a pipette pump on the proximal end of the pipette. The cartridge may also be disposable. Aspirator 10, or any other embodiment of the present disclosure, may have other kinds of cartridges, as hereinbelow described in detail. Additionally, a syringe may be attached to a proximal end of the cartridge or directly to a proximal end 22 of inner member 12 to collect blood or bone marrow of a vertebral body. The proximal end of the cartridge and the syringe may be attached to each other by a leur lock or any other suitable locking mechanism known in the art. Alternatively, the cartridge may include a reservoir at its proximal end to collect blood or bone marrow from a vertebral body, as seen in FIGS. 11A-11B and discussed in detail hereinbelow.

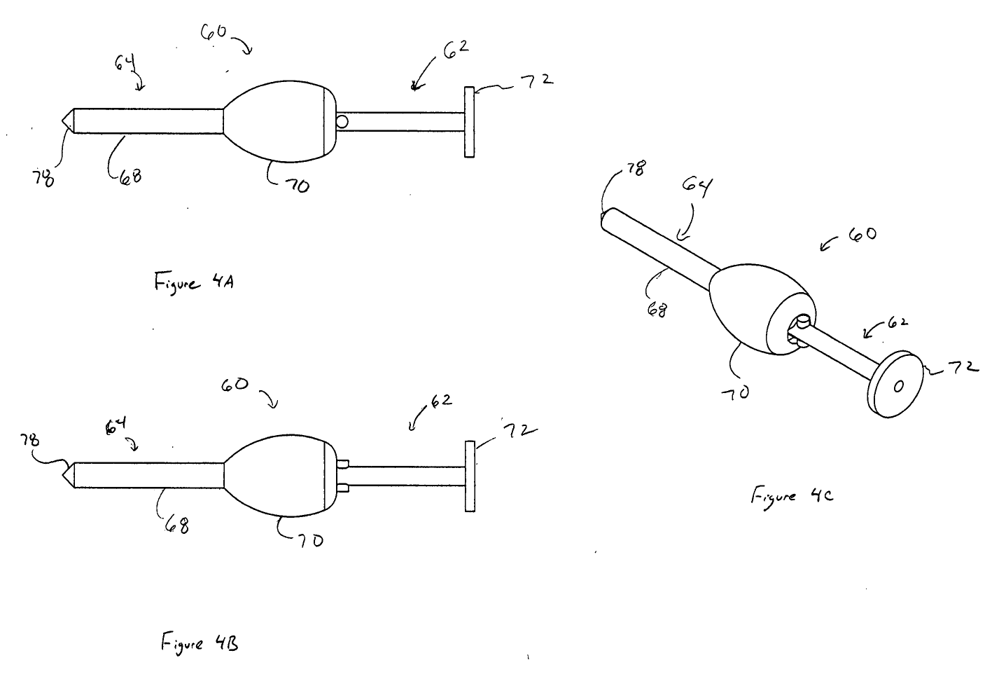

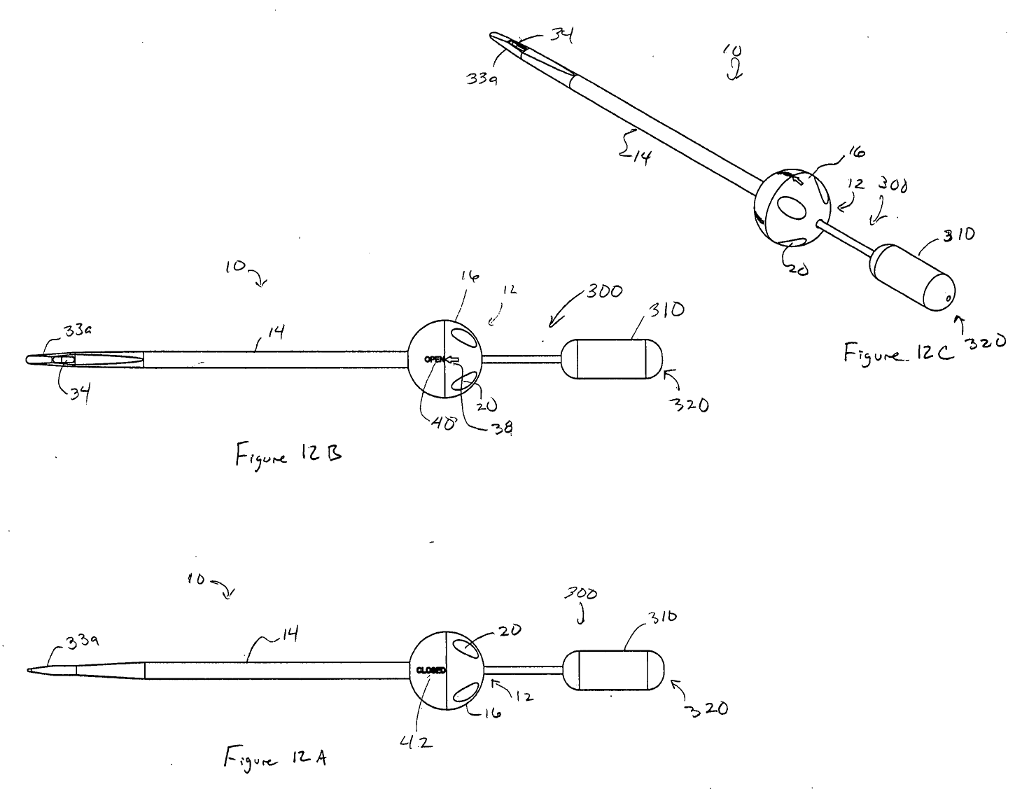

Referring to FIGS. 3A-3C, outer member 14 has a proximal end 28 and a distal end 30. The proximal end 28 of the outer member 14 has a support member for supporting the knob 16. The distal end 30 contains a tip 33 having an opening 34. Aspirator 10 may include an extended tip 33a, as illustrated in FIGS. 10A-10C. Additionally, outer member 14 includes a cannula 36 disposed therethrough. Cannula 36 is interconnected to opening 34.

As seen in FIGS. 1A-1C, knob 16 of inner member 12 may have a label 38 in the form of an arrow to indicate to a user where knob 16 has to be positioned to open or close opening 34. Support member 32 of outer member 14 may have two labels spelling the words “open” 40 and “closed” 42. An operator should align label 38 with the “open” label 40 to open opening 34 by rotating knob 16. To close opening 34, a surgeon has to align label 38 with “closed” label 42 by rotating knob 16.

To extract bodily fluids using aspirator 10, a surgeon must first find a vertebral body pedicle using a pedicle probe or any other suitable pedicle finder device. Aspirator 10, itself, may be designed to function as a pedicle probe. Alternatively, a separate pedicle probe may be positioned inside inner member 12. After locating the pedicle, the surgeon should introduce and position aspirator 10 in the desired extraction site. Aspirator 10 must be in the closed position before it is introduced into the human body to prevent extraction of unwanted substances. To position aspirator 10 in the closed position, the user must rotate knob 16 until the label 38 is aligned with the “closed” label 42. Once aspirator 10 is located in the desired position, the operator may open opening 34 by rotating knob 16. The operator should rotate knob 16 until label 38 is aligned with “open” label 40 to completely open opening 34. Tactile or audible “clicks” may be utilized to releasably maintain inner knob's 16 position relative to the outer knob 32. As soon as the user opens aspirator 10, negative pressure built up in the cartridge will force blood or bone marrow through opening 34. The blood or bone marrow will then travel through a cartridge and into a barrel of a syringe, a reservoir, or any other suitable collecting device known in the art.

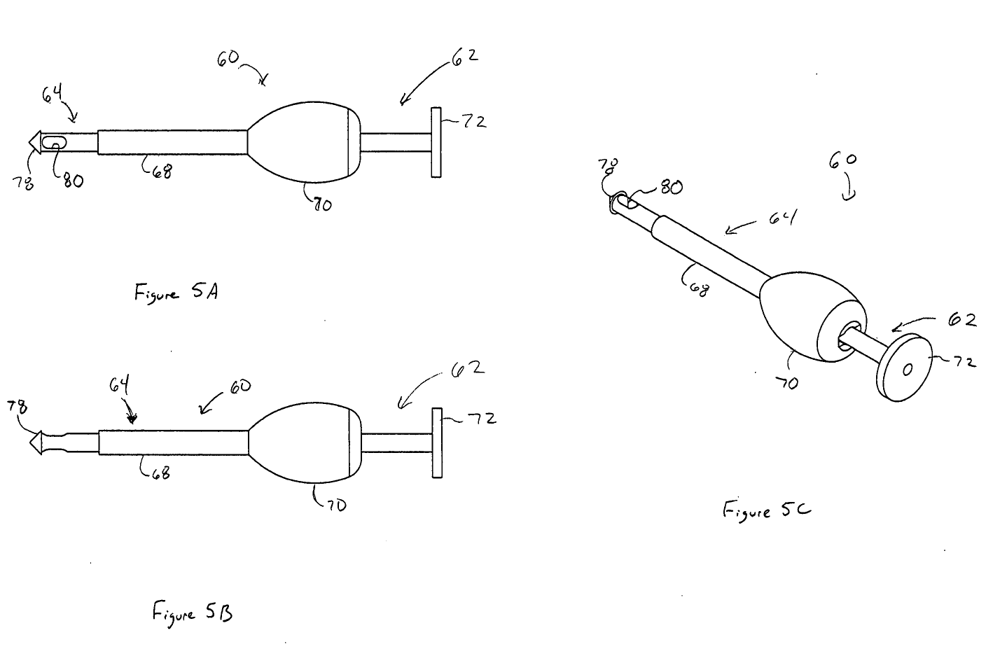

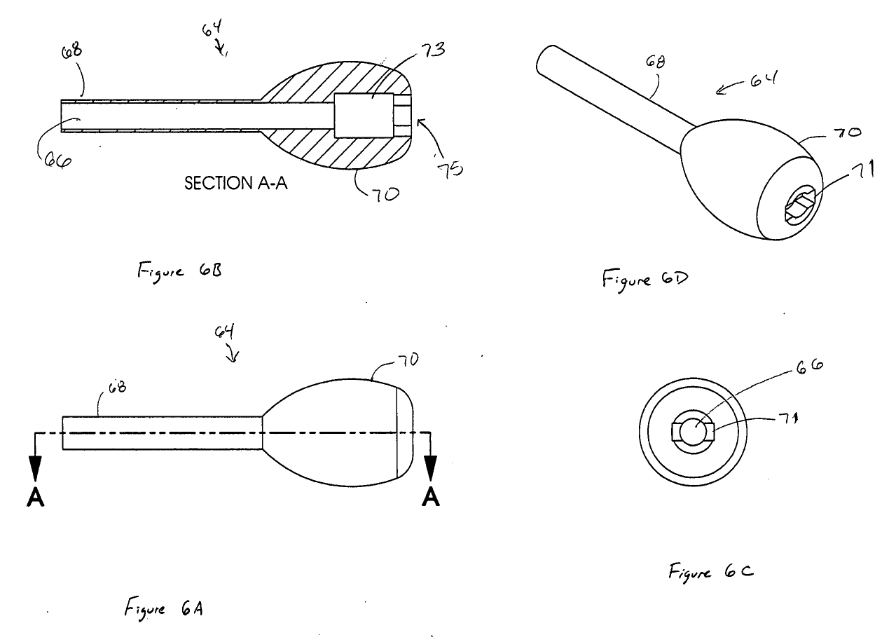

An embodiment of a translationally actuated aspirator according to the present disclosure is referred to in FIGS. 4A-4C as 60. Aspirator 60 includes an outer member 64 and an inner member 62 slidably and rotatably disposed within outer member 64. Outer member 64 includes a tubular member 68 and a support member 70 surrounding at least a portion of tubular member 68. Inner member 62 may include handle 72 located in a proximal end 74 thereof, a tip 78 positioned on the distal end 76 of inner member 62, pins 84 extending outwardly from inner member 62, and an opening 80 positioned on the distal end 76 of inner member 62. As illustrated in FIGS. 4A-4C, aspirator 60 may be place in a closed position. When aspirator 60 is in the closed position, opening 80 is contained within tubular member 68 of outer member 64. In the open position, opening 80 of aspirator 60 is located outside the tubular member 68 of outer member 64, as shown in FIGS. 5A-5C.

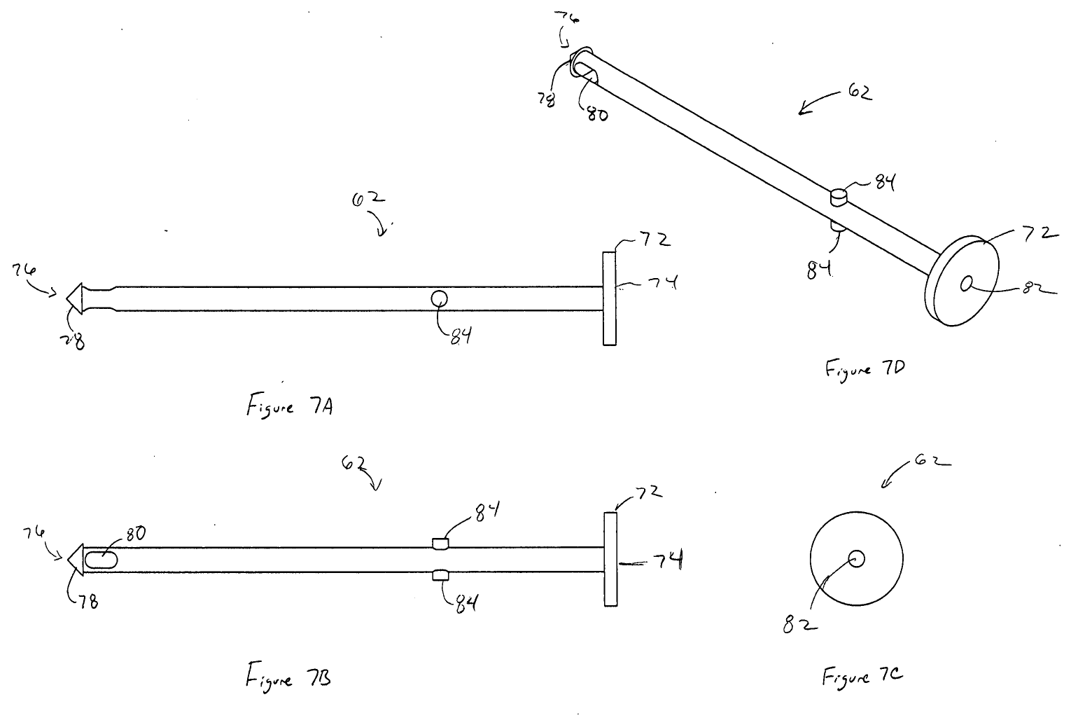

With reference to FIGS. 6A-6D, outer member 64 includes tubular member 68 having a cannula 66 disposed therethrough and dimensioned for receiving inner member 62. In addition, outer member 64 has a support member 70 surrounding at least a portion of tubular member 68 and a space 73. Space 73 is positioned inside support member 70 and is dimensioned for retaining pins 84. Support member 70 is located at the proximal end 75 of outer member 64. A slot 71 is positioned on the proximal end of support member 70 and is configured for allowing passage of pins 84. Specifically, pins 84 may be introduced into space 73 through slot 71 when they are coaxially aligned with slot 71.

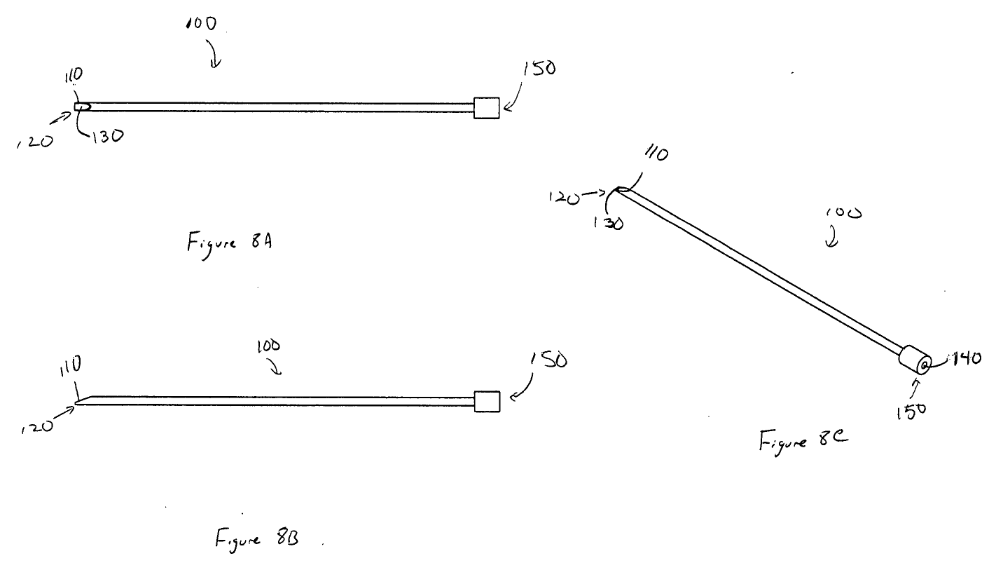

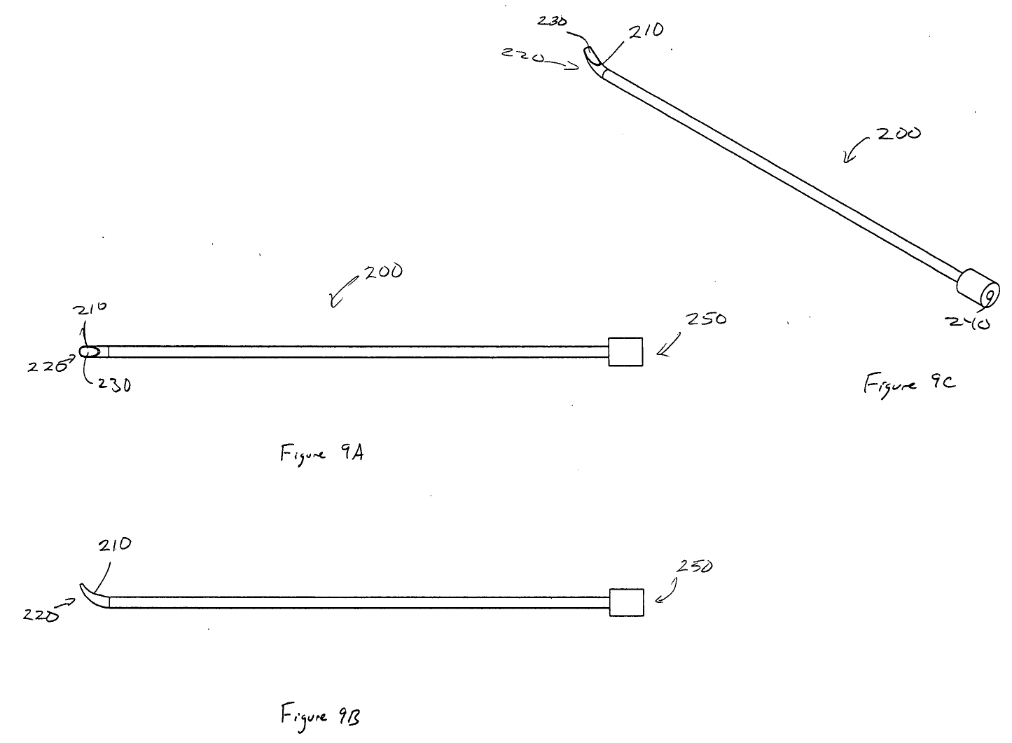

Referring now to FIGS. 7A-7D, inner member 62 has a proximal end 74 and a distal end 76. A handle 72 is attached to the proximal end 74 of inner member 62. The distal end 76 of inner member 62 has a tip 78 attached thereto. An opening 80 is positioned on the distal end 76 of the inner member 62. Additionally, inner member 62 has a cannula 82 disposed therethrough and it is dimensioned for receiving a cannulated cartridge. Cannula 82 and opening 80 are interconnected to one another. Pins 84 extend outwardly from the outer surface of inner member 62. Slot 71 of support member 70 is dimensioned for receiving pins 84. When aspirator 60 is in the open position, pins 84 are positioned inside space 73 of outer member 64. In the closed position, pins 85 of aspirator 60 are positioned outside outer member 64 and transverse to slot 71 to prevent distal translation of inner member 62. Although the figures show two pins, those skilled in the art will recognize that one or more pins may be used.

Aspirator 60 may be used to extract blood or bone marrow of vertebral bodies, or other similar bodily fluids. To extract such bodily fluids, a user must first locate a vertebral body pedicle using said device, a pedicle probe or any other suitable pedicle locating device known in the art. Then, while aspirator 60 is in its closed position, the operator must introduce and position aspirator 60 in the desired extraction site. Afterwards, the user must rotate inner member 62 until pins 84 fit into slot 71 and then advance axially inner member 62 by pushing handle 72. Once the user advances inner member 62, opening 80 is exposed and negative pressure will cause blood or bone marrow to travel through opening 80 and into a cartridge, a reservoir or any other suitable collecting device.

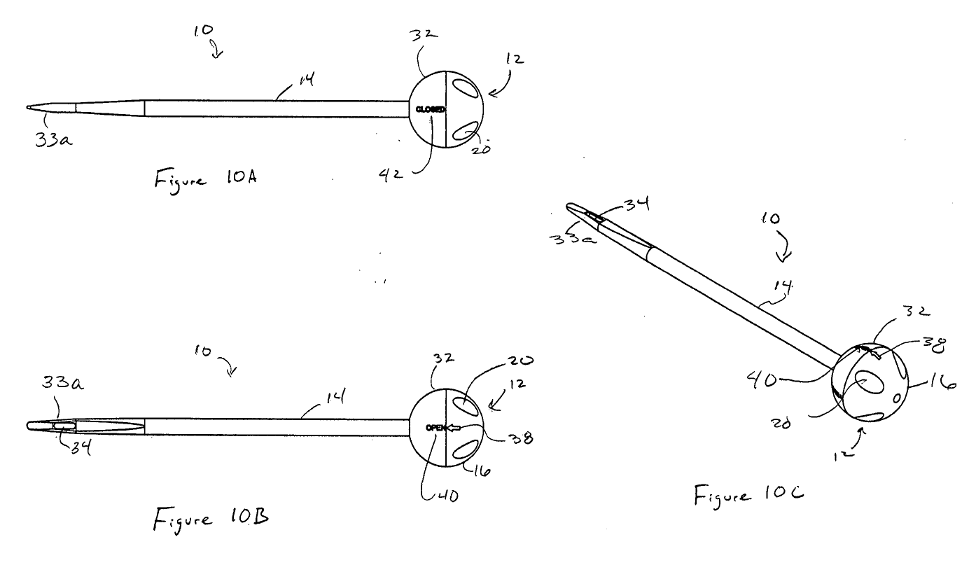

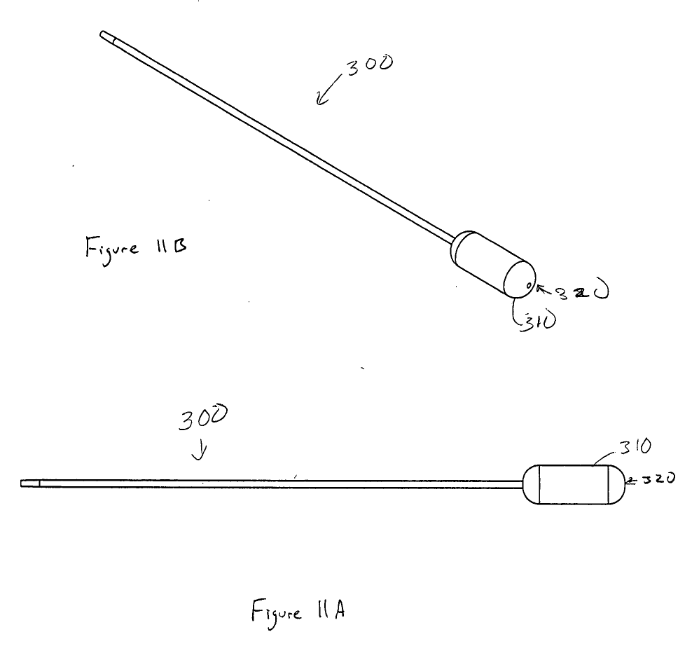

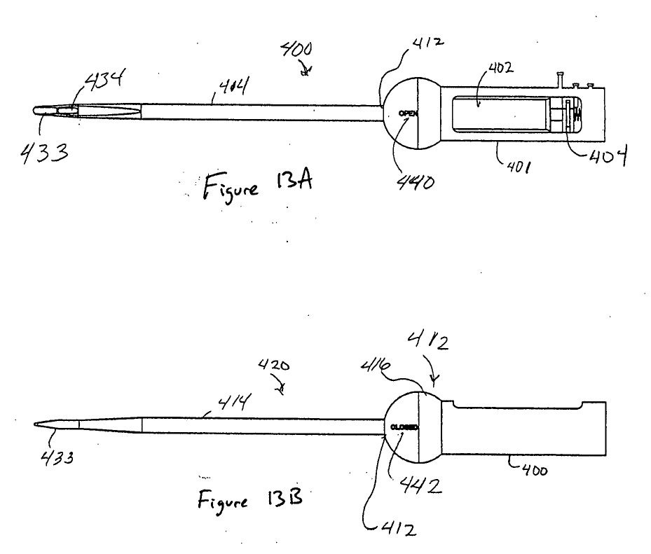

As discussed hereinabove, inner members 12, 62 of the the present disclosure are configured to receive a cartridge. Several kinds of cartridges may be placed within inner members 12, 62. For instance, as seen in FIGS. 8A-8C, an elongated cartridge 100 with a straight tip 110 at its distal end 120 may be inserted into inner members 12, 62. Tip 110 may include a hole 130. In addition, cartridge 100 may contain a cannula 140 disposed therethrough. A reservoir or a syringe may be attached to the proximal end 150 of cartridge 100. Alternatively, a cartridge 200 having a bent-tip 210 at its distal end 220 may be placed within the inner members 12, 62, as illustrated in FIGS. 9A-9C. Tip 210 includes a hole 230. Also, cartridge 200 includes a cannula 240 disposed therethrough. The proximal end 250 of the cartridge 200 may be connected to syringe through a leur lock. Otherwise, a cartridge 300 may have a reservoir 310 at its proximal end 320 to collect the blood or bone marrow, as shown in FIGS. 11A-11B. As seen in FIGS. 12A-12C, aspirator 10 may include a cartridge 300 having a reservoir 310 and an extended tip 33a. In this embodiment, cartridge 300 is partially disposed along cannula 26 of inner member 12. Reservoir 310, which is situated at the proximal end 320 of cartridge 300, is located outside of inner member 12 so that a user may easily access it and retrieve its contents. In still another embodiment, a cartridge may be in the form of a pipette (not shown). This embodiment may further include a pipette pump or other suitable pumping mechanism known in the art. The pipette may be placed within the inner member of an aspirator according to any of the embodiments disclosed herein.

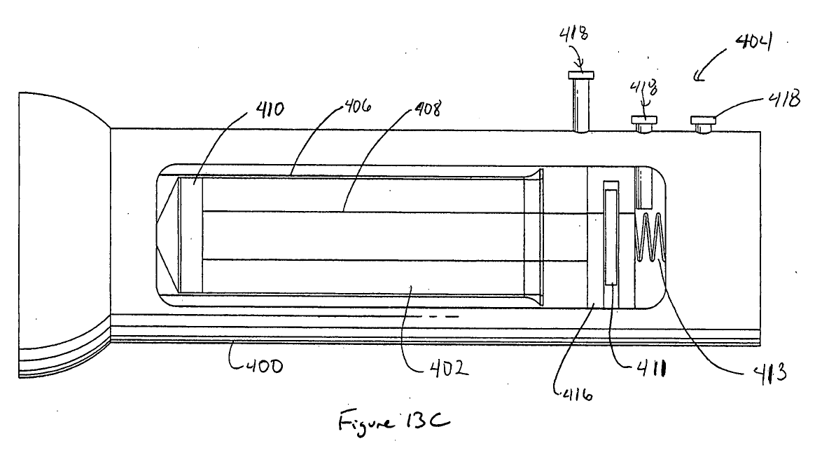

Any of the embodiments of the present disclosure may include a casing situated at it proximal end and capable of retaining a syringe. As shown in FIGS. 13A-13C, aspirator 400 may include casing 401 positioned at a proximal end 412 of aspirator 400, a syringe 402 supported within casing 401, and an automatic syringe actuator 404. As seen in FIG. 13C, syringe 402 includes a barrel 406 encompassing a plunger 408. Plunger 408 is slidably positioned within barrel 406, and includes a plunger tip 410 positioned at the distal end thereof and a plunger flange 411 attached to the proximal end thereof. Automatic syringe actuator 404 includes a spring 413 connected to a joint 416, and a plurality of cylindrical members 418 slidably mounted on the top surface of casing 400. Although the figures show three cylindrical members 418, a person skilled in the art will understand that automatic syringe actuator 404 may have one or more cylindrical members. The proximal end of spring 413 is attached to the proximal inner surface of casing 400 and the distal end of spring 413 is connected to joint 416. When piston 410 is advanced proximally, a negative pressure is created in barrel 406. Due to the negative pressure in barrel 406, syringe plunger 408 is biased proximally. Pins 418 prevent plunger 408 from advancing proximally against the force of spring 413. As pins 418 are moved outwardly, plunger 408 advances proximally to extract blood or bone marrow from a vertebral body.

In use, aspirator 400 is placed in the desired extraction site. A user must then rotate knob 416 of inner member to open hole 434 of outer member 414. Once hole 434 is opened, an operator may selectively move each pin 418 outwardly to displace syringe plunger 408 proximally and thereby extract a determined amount of vertebral blood or bone marrow. The negative pressure created in barrel 406 will force blood or bone marrow into hole 434 and into barrel 406.

The applications of the present disclosure are not limited to extraction of blood or bone morrow from vertebral bodies, but it may include any number of further extraction applications. It will be understood that various modifications may be made to the embodiments disclosed herein. Therefore, the above description should not be construed as limiting, but merely as exemplifications of various embodiments. Those skilled in the art will envision other modifications within the scope and spirit of the claims appended hereto.

What is claimed is:

1. An aspirator, comprising:
an inner member including a cannula disposed therethrough, the inner member having an opening at a distal end thereof and a rotatable knob at a proximal end thereof;
an outer member having a cannula disposed therethrough, the cannula of the outer member being configured and dimensioned for receiving the inner member, wherein the outer member has an aperture located at a distal end thereof; and
wherein the knob is configured to rotate the inner member with respect to the outer member and expose the opening of the inner member.

2. The aspirator according to claim 1, wherein the outer member includes an extended tip located at the distal end thereof.

3. The aspirator according to claim 1 further comprising a cartridge disposed within the inner member.

4. The aspirator according to claim 1, wherein the cartridge is disposable.

5. The aspirator according to claim 3, wherein the cartridge includes a straight-tip at a distal end thereof.

6. The aspirator according to claim 3, wherein the cartridge includes a bent-tip at a distal end thereof.

7. The aspirator according to claim 3, wherein the cartridge includes a reservoir attached to the proximal end thereof.

8. The aspirator according to claim 3, wherein a syringe is connected to the proximal end of the cartridge.

9. The aspirator according to claim 8, wherein the syringe is supported within a casing.

10. The device according to claim 8 further including an automatic syringe actuator connected at a proximal end of the syringe and configured to automatically operate the syringe, wherein the automatic syringe actuator includes a joint connected to the proximal end of the syringe, a plurality of pins slidably mounted on the casing, and a spring attached to the joint and the interior proximal surface of the casing.

11. The aspirator according to claim 1, wherein a reservoir is attached to the proximal end of the inner member.

12. The aspirator according to claim 1, wherein a cartridge is attached to the proximal end of the inner member.

13. The aspirator according to claim 1, wherein a syringe is attached to the proximal end of the inner member.

14. A aspirator, comprising:
an inner member having a cannula disposed therethrough, the inner member having an opening at a distal end thereof and a handle at a proximal end thereof;
an outer member having a cannula disposed therethrough, the cannula of the outer member being configured and dimensioned for receiving the inner member, wherein the outer member has an aperture located at a distal end thereof; and
wherein the inner member is configured to translate along the cannula of the outer member and expose the opening of the inner member.

15. The aspirator according to claim 14, wherein the outer member includes an extended tip located at the distal end thereof.

16. The aspirator according to claim 14 further comprising a cartridge disposed within the inner member.

17. The aspirator according to claim 16, wherein the cartridge includes a straight-tip at a distal end thereof.

18. The aspirator according to claim 16, wherein the cartridge includes a bent-tip at a distal end thereof.

19. The aspirator according to claim 16, wherein the cartridge includes a reservoir attached to the proximal end thereof.

20. The aspirator according to claim 16, wherein a syringe is connected to the proximal end of the cartridge.

21. The aspirator according to claim 20, wherein the syringe is supported within a casing.

22. The aspirator according to claim 20 further including an automatic syringe actuator connected at a proximal end of the syringe and configured to automatically operate the syringe, wherein the automatic syringe actuator includes a joint connected to the proximal end of the syringe, a plurality of pins slidably mounted on the casing, and a spring attached to the joint and the interior proximal surface of the casing.

23. The aspirator according to claim 14, wherein a reservoir is attached to the proximal end of the inner member.

24. The aspirator according to claim 14, wherein a cartridge is attached to the proximal end of the inner member.

25. The aspirator according to claim 14, wherein a syringe is attached to the proximal end of the inner member.

26. The method of extracting bodily fluids, comprising the steps of:
providing an aspirator including:
an inner member having a cannula disposed therethrough, the inner member having an opening at a distal end thereof and a rotatable knob at a proximal end thereof;
an outer member having a cannula disposed therethrough, the cannula of the outer member being configured and dimensioned for receiving the inner member, wherein the outer member has an aperture located at a distal end thereof; and
wherein the knob is configured to rotate inner member with respect to the outer member and expose the opening of the inner member;
finding a vertebral body pedicle;
placing the aspirator into the vertebral body pedicle; and
exposing the opening of the inner member; and
extracting bodily fluid.

27. The method of extracting bodily fluids, comprising the steps of:
providing an aspirator including:
an inner member having a cannula disposed therethrough, the inner member having an opening at a distal end thereof and a handle at a proximal end thereof;
an outer member having a cannula disposed therethrough, the cannula of the outer member being configured and dimensioned for receiving the inner member, wherein the outer member has an aperture located at a distal end thereof; and
wherein the inner member is configured to translate along the cannula of the outer member and expose the opening of the inner member;
finding a vertebral body pedicle;
placing the aspirator into the vertebral body pedicle; and
exposing the opening of the inner member.
